(19) United States
(12) Patent Application Publication
(10) Pub. No.: US 2022/0364410 A1
(43) Pub. Date: Nov. 17, 2022

(54) AUTOMATED ADJUSTMENT SYSTEM FOR NON-LIGHT-EMITTING VARIABLE TRANSMISSION DEVICES AND A METHOD OF USING THE SAME

(71) Applicant: Sage Electrochromics, Inc., Faribault, MN (US)

(72) Inventors: Leo SU, Lakeville, MN (US); Dallas Akre, Lonsdale, MN (US)

(21) Appl. No.: 17/663,197

(22) Filed: May 12, 2022

Related U.S. Application Data
(60) Provisional application No. 63/187,663, filed on May 12, 2021.

(51) Int. Cl. E06B 9/24; G02F 1/153; G02F 1/163; G02F 1/1514; A61B 5/0205; A61B 5/00

(52) U.S. Cl. CPC E06B 9/24; G02F 1/1533; G02F 1/163; G02F 1/1514; A61B 5/02055; A61B 5/4266; G02F 2001/15145; E06B 2009/2464; A61B 5/021

(57) ABSTRACT

A method of controlling a non-light emitting, variable transmission device is disclosed. The method can include receiving state information from at least one wearable device, prioritizing the received state information, sending signals from a remote management system to a first controller in response to the received prioritized state information, and changing a first transmission state of a non-light-emitting, variable transmission device to a second transmission state for the non-light-emitting, variable transmission device in response to the signals received from the first controller.

### CROSS-REFERENCE TO RELATED APPLICATION(S)

This application claims priority under 35 U.S.C. § 119(e) to U.S. Provisional Application No. 63/187,663, entitled “AUTOMATED ADJUSTMENT SYSTEM FOR NON-LIGHT-EMITTING VARIABLE TRANSMISSION DEVICES AND A METHOD OF USING THE SAME,” by Leo SU et al., filed May 12, 2021, which is assigned to the current assignee hereof and incorporated herein by reference in its entirety.

### FIELD OF THE DISCLOSURE

The present disclosure is directed to systems that include non-light-emitting variable transmission devices, and more specifically to systems including wearable devices, controllers, and non-light-emitting variable transmission devices and methods of using the same.

### BACKGROUND

A non-light-emitting variable transmission device can include an electrochromic stack where transparent conductive layers are used to provide electrical connections for the operation of the stack. Non-light-emitting variable transmission devices employ materials capable of reversibly altering their optical properties following electrochemical oxidation and reduction in response to an applied potential. The optical modulation is the result of the simultaneous insertion and extraction of electrons and charge compensating ions in the electrochemical material lattice.

The non-light-emitting variable transmission device can reduce glare and the amount of sunlight entering a room, thereby controlling the ambient temperature within a room. Buildings can include many non-light-emitting variable transmission devices that may be controlled locally (at each individual or a relatively small set of devices), for a room, or for a building (a relatively large set of devices). However, control for such devices relies on pre-calculated environmental data to anticipate the comfort levels within such a room or building. As such, a need exists for a better control strategy.

### BRIEF DESCRIPTION OF THE DRAWINGS

Embodiments are illustrated by way of example and are not limited in the accompanying figures.

FIG. 1 includes a schematic depiction of a system for controlling a set of non-light-emitting, variable transmission devices in accordance with an embodiment.

FIG. 2 includes an illustration of a top view of the substrate, the stack of layers, and the bus bars.

FIG. 3A includes an illustration of a cross-sectional view along line A of a portion of a substrate, a stack of layers for an electrochromic device, and bus bars, according to one embodiment.

FIG. 3B includes an illustration of a cross-sectional view along line B of a portion of a substrate, a stack of layers for an electrochromic device, and bus bars, according to one embodiment.

FIG. 4 includes a flow diagram for operating the system of FIG. 1 or 2.

Skilled artisans appreciate that elements in the figures are illustrated for simplicity and clarity and have not necessarily been drawn to scale. For example, the dimensions of some of the elements in the figures may be exaggerated relative to other elements to help to improve understanding of embodiments of the invention.

### DETAILED DESCRIPTION

The following description in combination with the figures is provided to assist in understanding the teachings disclosed herein. The following discussion will focus on specific implementations and embodiments of the teachings. This focus is provided to assist in describing the teachings and should not be interpreted as a limitation on the scope or applicability of the teachings.

The terms “normal operation” and “normal operating state” refer to conditions under which an electrical component or device is designed to operate. The conditions may be obtained from a data sheet or other information regarding voltages, currents, capacitances, resistances, or other electrical parameters. Thus, normal operation does not include operating an electrical component or device well beyond its design limits.

The term “color rendering,” when referring to an electrical device, is intended to refer to the color fidelity of a space to keep the color within the space within a wavelength of between 390 nm and 700 nm as a result of a light source or filter.

As used herein, the terms “comprises,” “comprising,” “includes,” “including,” “has,” “having,” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present), and B is false (or not present), A is false (or not present), and B is true (or present), and both A and B are true (or present).

The use of “a” or “an” is employed to describe elements and components described herein. This is done merely for convenience and to give a general sense of the scope of the invention. This description should be read to include one or at least one and the singular also includes the plural, or vice versa, unless it is clear that it is meant otherwise.

The use of the word “about,” “approximately,” or “substantially” is intended to mean that a value of a parameter is close to a stated value or position. However, minor differences may prevent the values or positions from being exactly as stated. Thus, differences of up to ten percent (10%) for the value are reasonable differences from the ideal goal of exactly as described.

Unless otherwise defined, all technical and scientific terms used herein have the same meaning as commonly understood by one of ordinary skill in the art to which this invention belongs. The materials, methods, and examples are illustrative only and not intended to be limiting. To the extent not described herein, many details regarding specific materials and processing acts are conventional and may be found in textbooks and other sources within the glass, vapor deposition, and electrochromic arts.

A system can include a non-light-emitting, variable transmission device; a wearable device to provide data for controlling the non-light-emitting variable transmission device, a controller coupled and configured to provide power to the non-light-emitting, variable transmission device; and a router configured to provide power and control signals to the controller.

The systems and methods are better understood after reading the specification in conjunction with the figures. System architectures are described and illustrated, followed by an exemplary construction of a non-light-emitting, variable transmission device, and a method of controlling the system. The embodiments described are illustrative and not meant to limit the scope of the present invention, as defined by the appended claims.

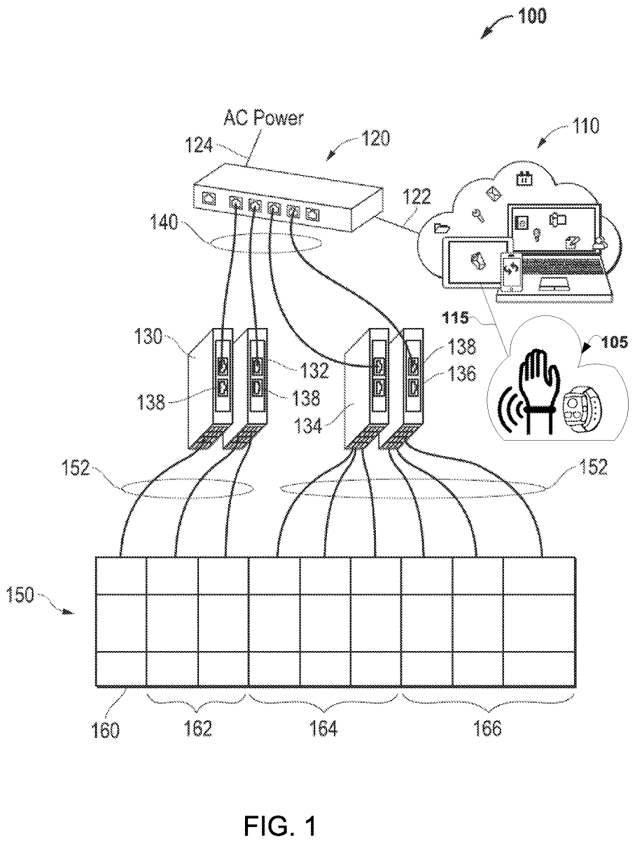

Referring to FIG. 1, a system for controlling a set of non-light-emitting, variable transmission devices is illustrated and is generally designated 100. As depicted, the system 100 can include a building management system 110. In a particular aspect, the building management system 110 can include a computing device such as a desk top computer, a laptop computer, a tablet computer, a smartphone, some other computing device, or a combination thereof. In one embodiment, the computing device can be connected to a wearable device 105 via a control link 115. The control link 115 can be a wireless connection. In an embodiment, the control link 115 can use a wireless local area network connection operating according to one or more of the standards within the IEEE 802.11 (WiFi) family of standards. In a particular aspect, the wireless connections can operate within the 2.4 GHz ISM radio band, within the 5.0 GHz ISM radio band, or a combination thereof. The computing device can be configured to analyze the data received from the wearable device and control the non-light-emitting variable transmission device based from the data received from the wearable device. The building management system 110 can be used to control the heating ventilation air condition (HVAC) system of the building, interior lighting, exterior lighting, emergency lighting, fire suppression equipment, elevators, escalators, alarms, security cameras, access doors, another suitable component or sub-system of the building, or any combination thereof.

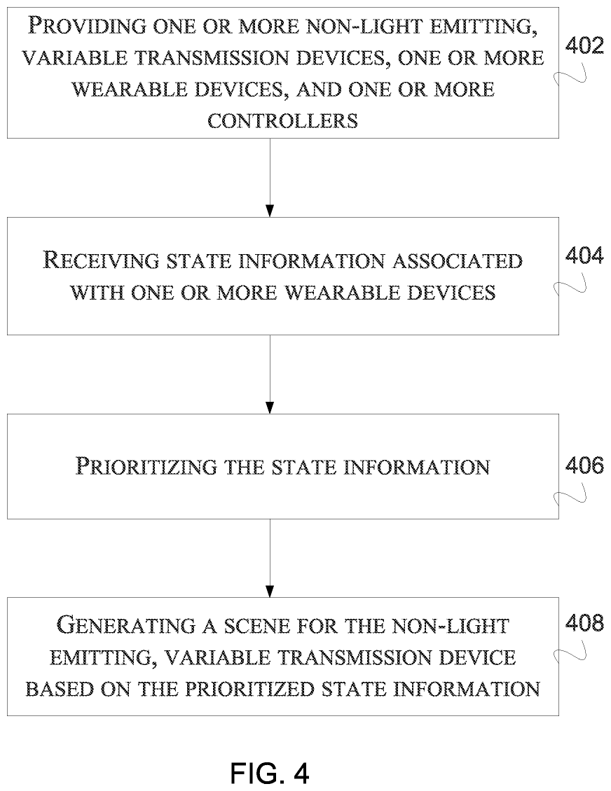

The wearable device 105 can be a watch, a ring, a band, a necklace, a hat, wearable glasses, an activity tracker, or any other wearable device 105. In one embodiment, the wearable device 105 can have a display element, a body that can be secured around a wearer, and circuitry for controlling the display element. In such an embodiment, the body can include a strap such as a wristband. In another embodiment, the wearable device 105 can be secured around an ankle, a leg, a finger, on a head, or around any other portion of a body. The wearable device 105 may include a power source such as a rechargeable battery. The wearable device 105 may include sensors that acquire physiological or pedometric data. The wearable device 105 can communicate with external devices, such as the building management system 110 to then in turn control the non-light-emitting variable transmission devices within a window frame panel 150. The method of operation is described in greater detail below in conjunction with FIG. 4.

As illustrated in FIG. 1, the system 100 can include a router 120 connected to the building management system 110 via a control link 122. The control link 122 can be a wireless connection. In an embodiment, the control link 122 can use a wireless local area network connection operating according to one or more of the standards within the IEEE 802.11 (WiFi) family of standards. In a particular aspect, the wireless connections can operate within the 2.4 GHz ISM radio band, within the 5.0 GHz ISM radio band, or a combination thereof.

Regardless of the type of control link 122, the building management system 110 can provide control signals to the router 120 via the control link 122. The control signals can be used to control the operation of one or more non-light-emitting variable transmission devices that are indirectly, or directly, connected to the router 120 and described in detail below. As indicated in FIG. 1, the router 120 can be connected to an alternating current (AC) power source 124. The router 120 can include an onboard AC-to-direct current (DC) converter (not illustrated). The onboard AC-to-DC converter can convert the incoming AC power from the AC power source 124, approximately 120 Volts (V) AC, to a DC voltage that is at most 60 VDC, 54 VDC, 48 VDC, 24 VDC, at most 12 VDC, at most 6 VDC, or at most 3 VDC.

FIG. 1 also indicates that the router 120 can include a plurality of connectors. The system 100 can include controllers 130, 132, 134, and 136 connected to the router 120. The router 120 can be configured to provide power and control signals to the controllers 130, 132, 134, and 136. Each of the controllers 130, 132, 134, and 136 can include a plurality of connectors 138. As illustrated in FIG. 1, a plurality of cables 140 can used to connect the controllers 130, 132, 134, and 136 to the router 120. Each of the cables 140 can include a Category 3 cable, a Category 5 cable, a Category 5e cable, a Category 6 cable, or another suitable cable. In another embodiment, each cable 140 can be configured to transmit at least 4 W of power, and in another embodiment, each cable can be configured to transmit at most 200 W of power. While the system 100 of FIG. 1 is illustrated with four controllers 130, 132, 134, and 136, the system 100 may include more or fewer controllers.

Still referring to FIG. 1, the system 100 can also include the window frame panel 150 electrically connected to the controllers 130, 132, 134, and 136 via a plurality of sets of frame cables 152. The window frame panel 150 can include a plurality of non-light-emitting, variable transmission devices, each of which may be connected to its corresponding controller via its own frame cable. In the embodiment as illustrated, the non-light-emitting, variable transmission devices are oriented in a 3×9 matrix. In another embodiment, a different number of non-light-emitting, variable transmission devices, a different matrix of the non-light-emitting, variable transmission devices, or both may be used. Each of the non-light-emitting, variable transmission devices may be on separate glazings. In another embodiment, a plurality of non-light-emitting, variable transmission devices can share a glazing. For example, a glazing may correspond to a column of non-light-emitting, variable transmission devices in FIG. 1. A glazing may correspond to a plurality of column of non-light-emitting, variable transmission devices. In another embodiment, a pair of glazings in the window frame panel 150 can have different sizes, such glazings can have a different number of non-light-emitting, variable transmission devices. After reading this specification, skilled artisans will be able to determine a particular number and organization of non-light-emitting, variable transmission devices for a particular application.

With respect to a configuration, the system 100 can include a logic element, e.g., within the management system 110 that can perform the method steps described in FIG. 4. In particular, the logic element can be configured to determine power requirements for the controllers based on the data received from the wearable device 105 to change the ambient environment within a room or building. The system can be used with a wide variety of different types of non-light-emitting variable transmission devices. The apparatuses and methods can be implemented with switchable devices that affect the transmission of light through a window. Much of the description below addresses embodiments in which the switchable devices are electrochromic devices. In other embodiments, the switchable devices can include suspended particle devices, liquid crystal devices that can include dichroic dye technology, and the like. Thus, the concepts as described herein can be extended to a variety of switchable devices used with windows.

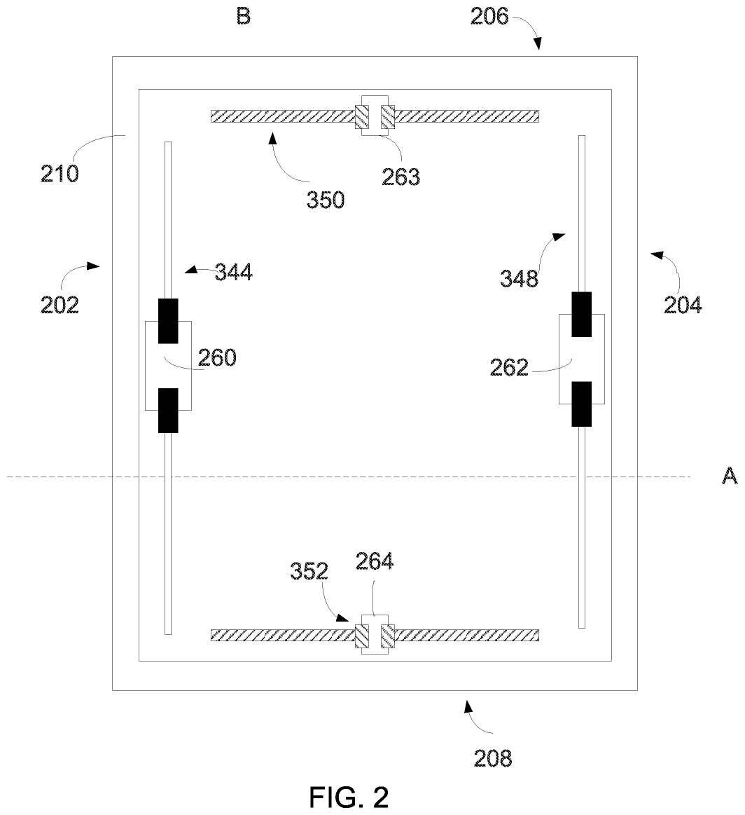

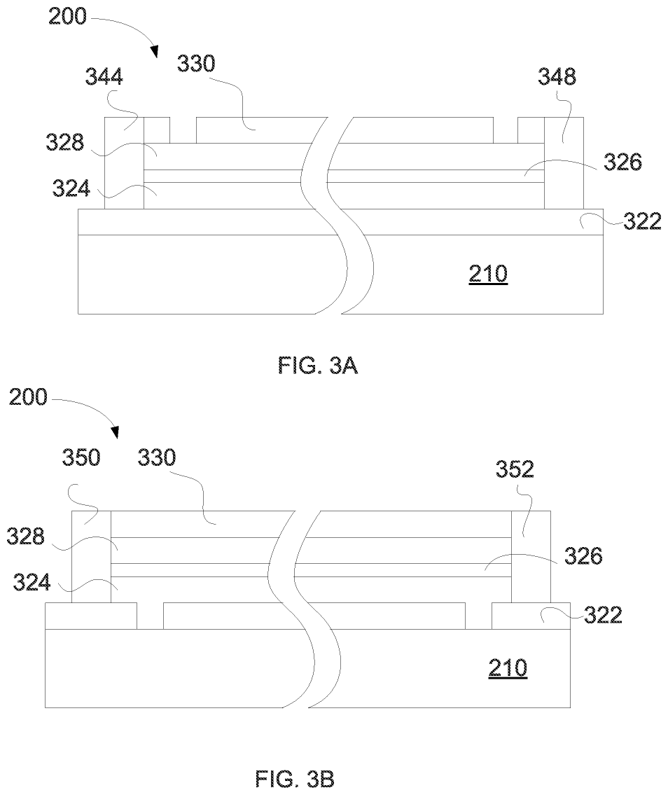

The description with respect to FIGS. 2, 3A, and 3B provide exemplary embodiments of a glazing that includes a glass substrate and a non-light-emitting variable transmission device disposed thereon. The embodiment as described with respect to FIGS. 2, 3A, and 3B is not meant to limit the scope of the concepts as described herein. In the description below, a non-light-emitting variable transmission device will be described as operating with voltages on bus bars being in a range of 0 V to 3 V. Such description is used to simplify concepts as described herein. Other voltage may be used with the non-light-emitting variable transmission device or if the composition or thicknesses of layers within an electrochromic stack are changed. The voltages on bus bars may both be positive (1 V to 4 V), both negative (−5 V to −2 V), or a combination of negative and positive voltages (−1 V to 2 V), as the voltage difference between bus bars are more important than the actual voltages. Furthermore, the voltage difference between the bus bars may be less than or greater than 3 V. After reading this specification, skilled artisans will be able to determine voltage differences for different operating modes to meet the needs or desires for a particular application. The embodiments are exemplary and not intended to limit the scope of the appended claims.

FIG. 2 is an illustration of a top view of a substrate 200, a stack of layers of an electrochromic device 322, 324, 326, 328, and 330, and bus bars 344, 348, 350, and 352 overlying the substrate 300, according to one embodiment. In an embodiment, the substrate 210 can include a glass substrate, a sapphire substrate, an aluminum oxynitride substrate, or a spinel substrate. In another embodiment, the substrate 210 can include a transparent polymer, such as a polyacrylic compound, a polyalkene, a polycarbonate, a polyester, a polyether, a polyethylene, a polyimide, a polysulfone, a polysulfide, a polyurethane, a polyvinylacetate, another suitable transparent polymer, or a co-polymer of the foregoing. The substrate 210 may or may not be flexible. In a particular embodiment, the substrate 210 can be float glass or a borosilicate glass and have a thickness in a range of 0.5 mm to 4 mm thick. In another particular embodiment, the substrate 210 can include ultra-thin glass which is a mineral glass having a thickness in a range of 50 microns to 300 microns. In a particular embodiment, the substrate 210 may be used for many different non-light-emitting variable transmission devices being formed and may be referred to as a motherboard.

The bus bar 344 lies along a side 202 of the substrate 210 and the bus bar 348 lies along a side 204 that is opposite the side 202. The bus bar 350 lies along side 206 of the substrate 210, and the bus bar 352 lies along side 208 which is opposite side 206. Each of the bus bars 344, 348, 350, and 352 have lengths that extend a majority of the distance each side of the substrate. In a particular embodiment, each of the bus bars 344, 348, 350, and 352 have a length that is at least 75%, at least 90%, or at least 95% of the distance between the sides 202, 204, 206, and 208, respectively. The lengths of the bus bars 344 and 348 are substantially parallel to each other. As used herein, substantially parallel is intended to mean that the lengths of the bus bars 344 and 348, 350 and 352 are within 10 degrees of being parallel to each other. Along the length, each of the bus bars has a substantially uniform cross-sectional area and composition. Thus, in such an embodiment, the bus bars 344, 348, 350, and 352 have a substantially constant resistance per unit length along their respective lengths.

In one embodiment, the bus bar 344 can be connected to a first voltage supply terminal 260, the bus bar 348 can be connected to a second voltage supply terminal 262, the bus bar 350 can be connected to a third voltage supply terminal 263, and the bus bar 352 can be connected to a fourth voltage supply terminal 264. In one embodiment, the voltage supply terminals can be connected to each bus bar 344, 348, 350, and 352 about the center of each bus bar. In one embodiment, each bus bar 344, 348, 350, and 352 can have one voltage supply terminal. The ability to control each voltage supply terminal 260, 262, 263, and 264 provides for control over grading of light transmission through the electrochromic device 124.

In one embodiment, the first voltage supply terminal 260 can set the voltage for the bus bar 344 at a value less than the voltage set by the voltage supply terminal 263 for the bus bar 350. In another embodiment, the voltage supply terminal 263 can set the voltage for the bus bar 350 at a value greater than the voltage set by the voltage supply terminal 264 for the bus bar 352. In another embodiment, the voltage supply terminal 263 can set the voltage for the bus bar 350 at a value less than the voltage set by the voltage supply terminal 264 for the fourth bus bar 352. In another embodiment, the voltage supply terminal 260 can set the voltage for the bus bar 344 at a value about equal to the voltage set by the voltage supply terminal 262 for the bus bar 348. In one embodiment, the voltage supply terminal 260 can set the voltage for the bus bar 344 at a value within about 0.5V, such as 0.4V, such as 0.3V, such as 0.2V, such as 0.1V to the voltage set by the voltage supply terminal 262 for the second bus bar 348. In a non-limiting example, the first voltage supply terminal 260 can set the voltage for the bus bar 344 at 0V, the second voltage supply terminal 262 can set the voltage for the bus bar 348 at 0V, the third voltage supply terminal 263 can set the voltage for the bus bar 350 at 3V, and the fourth voltage supply terminal 264 can set the voltage for the bus bar 352 at 1.5V.

The compositions and thicknesses of the layers are described with respect to FIGS. 3A and 3B. Transparent conductive layers 322 and 330 can include a conductive metal oxide or a conductive polymer. Examples can include a tin oxide or a zinc oxide, either of which can be doped with a trivalent element, such as Al, Ga, In, or the like, a fluorinated tin oxide, or a sulfonated polymer, such as polyaniline, polypyrrole, poly(3,4-ethylenedioxythiophene), or the like. In another embodiment, the transparent conductive layers 322 and 330 can include gold, silver, copper, nickel, aluminum, or any combination thereof. The transparent conductive layers 322 and 330 can have the same or different compositions.

The set of layers further includes an electrochromic stack that includes the layers 324, 326, and 328 that are disposed between the transparent conductive layers 322 and 330. The layers 324 and 328 are electrode layers, wherein one of the layers is an electrochromic layer, and the other of the layers is an ion storage layer (also referred to as a counter electrode layer). The electrochromic layer can include an inorganic metal oxide electrochemically active material, such as WO₃, V₂O₅, MoO₃, Nb₂O₅, TiO₂, CuO, Ir₂O₃, Cr₂O₃, CO₂O₃, Mn₂O₃, or any combination thereof and have a thickness in a range of 50 nm to 2000 nm. The ion storage layer can include any of the materials listed with respect to the electrochromic layer or Ta₂O₅, ZrO₂, HfO₂, Sb₂O₃, or any combination thereof, and may further include nickel oxide (NiO, Ni₂O₃, or combination of the two), and Li, Na, H, or another ion and have a thickness in a range of 80 nm to 500 nm. An ion conductive layer 326 (also referred to as an electrolyte layer) is disposed between the electrode layers 324 and 328 and has a thickness in a range of 20 microns to 60 microns. The ion conductive layer 326 allows ions to migrate therethrough and does not allow a significant number of electrons to pass therethrough. The ion conductive layer 326 can include a silicate with or without lithium, aluminum, zirconium, phosphorus, boron; a borate with or without lithium; a tantalum oxide with or without lithium; a lanthanide-based material with or without lithium; another lithium-based ceramic material; or the like. The ion conductive layer 326 is optional and, when present, may be formed by deposition or, after depositing the other layers, reacting portions of two different layers, such as the electrode layers 324 and 328, to form the ion conductive layer 326. After reading this specification, skilled artisans will appreciate that other compositions and thicknesses for the layers 322, 324, 326, 328, and 330 can be used without departing from the scope of the concepts described herein.

The layers 322, 324, 326, 328, and 330 can be formed over the substrate 210 with or without any intervening patterning steps, breaking vacuum, or exposing an intermediate layer to air before all the layers are formed. In an embodiment, the layers 322, 324, 326, 328, and 330 can be serially deposited. The layers 322, 324, 326, 328, and 330 may be formed using physical vapor deposition or chemical vapor deposition. In a particular embodiment, the layers 322, 324, 326, 328, and 330 are sputter deposited.

In the embodiment illustrated in FIGS. 3A and 3B, each of the transparent conductive layers 322 and 330 include portions removed so that the bus bars 344/348 and 350/352 are not electrically connected to each other. Such removed portions are typically 20 nm to 2000 nm wide. In a particular embodiment, the bus bars 344 and 348 are electrically connected to the electrode layer 324 via the transparent conductive layer 322, and the bus bars 350 and 352 are electrically connected to the electrode layer 328 via the transparent conductive layer 330. The bus bars 344, 348, 350, and 352 include a conductive material. In an embodiment, each of the bus bars 344, 348, 350, and 352 can be formed using a conductive ink, such as a silver frit, which is printed over the transparent conductive layer 322. In another embodiment, one or both of the bus bars 344, 348, 350, and 352 can include a metal-filled polymer. In a particular embodiment (not illustrated), the bus bars 350 and 352 are each a non-penetrating bus bar that can include the metal-filled polymer that is over the transparent conductive layer 330 and spaced apart from the layers 322, 324, 326, and 328. The viscosity of the precursor for the metal-filled polymer may be sufficiently high enough to keep the precursor from flowing through cracks or other microscopic defects in the underlying layers that might be otherwise problematic for the conductive ink. The lower transparent conductive layer 322 does not need to be patterned in this particular embodiment. In one embodiment, bus bars 344 and 348 are opposed each other. In one embodiment, bus bars 350 and 352 are orthogonal to bus bar 344.

In the embodiment illustrated, the width of the non-light-emitting variable transmission device W_{EC} is a dimension that corresponds to the lateral distance between the removed portions of the transparent conductive layers 322 and 330. W_{S} is the width of the stack between the bus bars 344 and 348. The difference in W_{S} and W_{EC} is at most 5 cm, at most 2 cm, or at most 0.9 cm. Thus, most of the width of the stack corresponds to the operational part of the non-light-emitting variable transmission device that allows for different transmission states. In an embodiment, such operational part is the main body of the non-light-emitting variable transmission device and can occupy at least 90%, at least 95%, at least 98% or more of the area between the bus bars 344 and 348.

Attention is now addressed to installing, configuring, and using the system as illustrated in FIG. 1 with glazings and non-light-emitting, variable transmission devices that can be similar to the glazing and non-light-emitting, variable transmission device as illustrated and described with respect to FIGS. 2, 3A, and 3B. In another embodiment, other designs of glazings and non-light-emitting, variable transmission devices.

FIG. 4 includes flow chart for a method 400 of operating the system 100 illustrated in FIG. 1. Commencing at block 402, the method can include providing one or more non-light-emitting, variable transmission devices, one or more wearable devices, and one or more controllers coupled to the one or more non-light emitting, variable transmission devices. In an embodiment, the non-light-emitting, variable transmission devices, wearable devices, and controllers may be connected to each other as illustrated in FIG. 1 and use non-light-emitting variable transmission devices similar to the non-light-emitting variable transmission device described and illustrated in FIGS. 2, 3A, and 3B.

Continuing the description of the method 400, at block 404, the method can include receiving state information associated with the non-light emitting, variable transmission devices of the glazing. In one embodiment, the management system 110 can send a constant signal to detect the presence of a wearable device on the network. In another embodiment, the wearable device sends a signal to the management system 110 to connect the two together. Once connected, the management system 110 can receive state information from the wearable device 105.

The collection of state information may occur nearly continuously, such as from a motion sensor, light sensor, or the like, on a periodic basis, such as once a minute, every ten minutes, hourly, or the like, or a combination thereof. This state information can be received at the router 120. This state information may be contained within the one or more wearable devices 105. In another embodiment, the state information can be contained within a look-up table provided in conjunction with these wearable devices 105, information provided by the building management system 110, or an external source. Alternatively, the state information can be based off of a simulation or 3D model algorithm that anticipates the conditions of the non-light emitting, variable transmission device. This state information can be manually input into a building management system, and the building management system 110 can push this information to the router 120 while the system 100 is being initially configured, reconfigured, during normal operation, or during a system reboot.

In one embodiment, an I/O unit can be coupled to the control devices 130, 132, 134, and 136 through the router 120. The state information can include a physiological change, a temperature, heart rate, frequency of respiration or blood oxygenation levels, blood pressure, such as systolic and diastolic numbers, body temperature, perspiration, and the like. The state information can also include, a sun position, weather conditions, a time of day, and a calendar day. In yet another embodiment, the state information can include an elapsed time since a scene has been changed, heat load within the controlled space, a contrast level between relatively bright and relatively dark objects within a field of view where an occupant is normally situated within the controlled space, whether an orb of the sun is in the field of view where the occupant is normally situated within the controlled space, whether a reflection of the sun is in the field of view where the occupant is normally situated within the controlled space, a level of cloudiness, or another suitable parameter, or any combination thereof. The state information may be collected at the I/O unit from sources of state information, such as sensors, a calendar, a clock, a weather forecast, or the like. The controlled space can be an area surrounding a window of the EC device or a space within a building. The controlled space may be a room, such as a meeting room or an office, or may be part of a floor of a building. The EC device can then affect light, glare, or temperature of the controlled space.

The building management system 110 can include logic to control the operation of building environmental and facility controls, such as heating, ventilation, and air conditioning (HVAC), lights, scenes for EC devices, including the EC device 200. The logic for the building management systems 110 can be in the form of hardware, software, or firmware. In an embodiment, the logic may be stored in a field programmable gate array (FPGA), an application-specific integrated circuit (ASIC), a hard drive, a solid state drive, or another persistent memory. In an embodiment, the building management system 110 may include a processor that can execute instructions stored in memory within the building management system 110 or received from an external source. In one embodiment, the external source can include one or more wearable devices. In another embodiment, the external source can be a combination of one or more wearable devices and other sensors, such as a rooftop sensor or one or more devices that include 360 degree sensors. By combining the data from the plurality of sensors, and the one or more wearable devices, the building management system can receive data from both interior and exterior the space.

After receiving the state information, the I/O unit can include logic to categorize and prioritize the state information, at block 406. In one embodiment, the state information can be included into at least two categories. In another embodiment, the state information can be included into at least three categories and no more than twenty categories. For example, the categories can include comfort, temperature control, glare control, daylight transmission, color rendering, and energy saving. The prioritization of the categories can be assigned based on criteria set prior to installation of the non-light-emitting, variable transmission devices. In one embodiment, the data from the wearable device may be prioritization above any other sensor.

The state information may be used to send instructions to control devices 130, 132, 134, and 136. The system 100 can be used to allow for scene-based control of EC device within a window, such as an IGU installed as part of architectural glass along a wall of a building or a skylight, or within a vehicle. The method 400 can include generating a scene for a window, at block 408. A few exemplary scenes can include all EC devices for a window being at the highest transmission state (fully tinted), all EC devices for the window being at the lowest transmission state (bleached), and different rows of EC devices for the window being at other transmission states. In one embodiment, a scene can include a graded transmission. The transmission information may be for each EC device within a scene, so that the scene may be recreated at a later time. At a time after generating the original scenes, an occupant or facilities personnel may save a scene that the he or she particularly likes or generates. Such a scene is referred to as a learned scene. For example, after a physical configuration of the controlled space is changed, new scenes may be generated that are more appropriate for the new physical configuration. The local control devices 130, 132, 134, and 136 can include a button that allows the occupant or another human to provide input to the apparatus 200 via the I/O unit to store the scene. The local control devices 130, 132, 134, and 136 may include another button that allows the occupant or another human to provide input to the apparatus 200 via the I/O unit to delete or invalidate the scene. Still further, the local control devices 130, 132, 134, and 136 may allow the occupant to adjust individual EC devices or subsets of EC devices and save the particular scene created. Yet further, when the occupant changes, the learned scenes may be deleted, and the original scenes restored. Learned scenes may be assigned a higher preference or weighing factor.

The scene selection may be correlated with and based on the prioritization of the state information. The method can include adding the scene to the collection of scenes. On a subsequent day, the control devices 130, 132, 134, and 136 may later select an original or learned scene from the collection of scenes when such scene's corresponding state information matches or is close to state information at the time when the control devices 130, 132, 134, and 136 is being used to select a scene. After reading this specification, skilled artisans will understand that the order of actions in FIG. 4 may be changed. Furthermore, one or more actions may not be performed, and one or more further actions may be performed in generating the collection of scenes. After the collection of scenes is generated, a scene from the collection can be selected, and a control device can control the EC devices of the window to achieve scene for the window.

A decision may be performed to determine whether there is a significant change in the state information, at an additional step in method 400. For example, a person may have entered the controlled space that was previously unoccupied with a second wearable device that includes additional state information related to physiologic changes. When the change is significant, the method can begin again to determine if the scene for the window should be changed.

Embodiments as described above can provide benefits over other systems with non-light-emitting, variable transmission devices. The use of remote scene selection and control can help with maintenance of an installed device. The methods as described herein allow all non-light-emitting, variable transmission devices coupled to be controlled individually based on the state information received and prioritization of that state information.

Many different aspects and embodiments are possible. Some of those aspects and embodiments are described below. After reading this specification, skilled artisans will appreciate that those aspects and embodiments are only illustrative and do not limit the scope of the present invention. Exemplary embodiments may be in accordance with any one or more of the ones as listed below.

Embodiment 1. A system, including: one or more wearable devices configured to generate state information; one or more non-light emitting, variable transmission devices; a remote management system configured to prioritize the state information and send a prioritized state information; and a control device configured to change a transmission state for the one or more non-light emitting, variable transmission devices in response to receiving the prioritized state information.

Embodiment 2. A system, including: a first non-light-emitting, variable transmission device; a first wearable device; a first controller coupled and configured to select a first scene from a collection of scenes for a non-light emitting, variable transmission device; and a management system that includes a logic element configured to: receive state information; prioritize the received state information; and send signals to the first controller in response to input corresponding to prioritized state information.

Embodiment 3. A method of controlling a non-light emitting, variable transmission device, including: receiving state information from at least one wearable device; prioritizing the received state information; and sending signals from a remote management system to a first controller in response to the received prioritized state information; changing a first transmission state of a non-light-emitting, variable transmission device to a second transmission state for the non-light-emitting, variable transmission device in response to the signals received from the first controller.

Embodiment 4. The method or system of any one of embodiments 1 to 3, where the remote management system is a wireless system.

Embodiment 5. The method of embodiment 3, further including sending a signal to the at least one wearable device for permission to access the state information stored on the at least one wearable device.

Embodiment 6. The system of embodiment 1, further including at least one non-light-emitting, variable transmission device, where: the non-light-emitting, variable transmission device comprise a first electrochromic device having a first edge, a second electrochromic device having a second edge, and a third electrochromic device having a third edge and a fourth edge; the first edge of the first electrochromic device is immediately adjacent to the third edge of the third electrochromic device, and the second edge of the second electrochromic device is immediately adjacent to the fourth edge of the third electrochromic device; and for the first scene, where comparing transmission levels of the first, second, and third electrochromic devices, the first electrochromic device has a lowest transmission level, the second electrochromic device has a graded transmission level, and the third electrochromic device has a highest transmission level.

Embodiment 7. The method or system of any one of embodiments 1 to 3, where changing the transmission state of the non-light-emitting, variable transmission device comprises changing from a first state to a second state, where the first state is full tint and the second state is a graded transmission level.

Embodiment 8. The method or system of any one of embodiments 1 to 3, where changing the transmission state of the non-light-emitting, variable transmission device comprises changing from a first state to a second state, where the first state is full clear and the second state is a graded transmission level.

Embodiment 9. The method or system of any one of embodiments 1 to 3, where changing the transmission state of the non-light-emitting, variable transmission device comprises changing from a first state to a second state, where the first state is full tint and the second state is a full clear transmission.

Embodiment 10. The method or system of any one of embodiments 1 to 3, where changing the transmission state of the non-light-emitting, variable transmission device comprises changing from a first state to a second state, where the first state is a graded transmission level and the second state is a fully tinted transmission level.

Embodiment 11. The method or system of any one of embodiments 1 to 3, where the prioritized state information comprises a blood pressure, heartbeat, perspiration, respiration frequency, and body temperature.

Embodiment 12. The method or system of any one of embodiments 1 to 3, where the non-light-emitting, variable transmission device includes: a first transparent conductive layer; a second transparent conductive layer; a cathodic electrochemical layer between the first transparent conductive layer and the second transparent conductive layer; and an anodic electrochemical layer between the first transparent conductive layer and the second transparent conductive layer.

Embodiment 13. The method or system of embodiment 12, where the non-light-emitting, variable transmission device further includes a substrate, where the first transparent conductive layer is on the substrate.

Embodiment 14. The method or system of embodiment 13, where the substrate includes glass, sapphire, aluminum oxynitride, spinel, polyacrylic compound, polyalkene, polycarbonate, polyester, polyether, polyethylene, polyimide, polysulfone, polysulfide, polyurethane, polyvinylacetate, another suitable transparent polymer, co-polymer of the foregoing, float glass, borosilicate glass, or any combination thereof.

Embodiment 15. The method or system of embodiment 12, where the cathodic electrochemical layer includes WO₃, V₂O₅, MoO₃, Nb₂O₅, TiO₂, CuO, Ni₂O₃, NiO, Ir₂O₃, Cr₂O₃, Co₂O₃, Mn₂O₃, mixed oxides (e.g., W—Mo oxide, W—V oxide), lithium, aluminum, zirconium, phosphorus, nitrogen, fluorine, chlorine, bromine, iodine, astatine, boron, a borate with or without lithium, a tantalum oxide with or without lithium, a lanthanide-based material with or without lithium, another lithium-based ceramic material, or any combination thereof.

Embodiment 16. The method or system of embodiment 12, further including an ion-conducting layer between the cathodic electrochemical layer and the anodic electrochemical layer.

Embodiment 17. The method or system of embodiment 16, where the ion-conducting layer includes lithium, sodium, hydrogen, deuterium, potassium, calcium, barium, strontium, magnesium, oxidized lithium, Li₂WO₄, tungsten, nickel, lithium carbonate, lithium hydroxide, lithium peroxide, or an alkaline earth metal, transition metal, Zn, Ga, Ge, Al, Cd, In, Sn, Sb, Pb, Bi, B, Si, P, S, As, Se, Te, silicates, silicon oxides, tungsten oxides, tantalum oxides, niobium oxides, borates, aluminum oxides, lithium silicate, lithium aluminum silicate, lithium aluminum borate, lithium aluminum fluoride, lithium borate, lithium nitride, lithium zirconium silicate, lithium niobate, lithium borosilicate, lithium phosphosilicate, other lithium-based ceramic materials, lithium salts, and dopants including lithium, sodium, hydrogen, deuterium, potassium, calcium, barium, strontium, magnesium, or combinations thereof.

Embodiment 18. The method or system of embodiment 12, where the second transparent conductive layer includes indium oxide, indium tin oxide, doped indium oxide, tin oxide, doped tin oxide, zinc oxide, doped zinc oxide, ruthenium oxide, doped ruthenium oxide and any combination thereof.

Embodiment 19. The method or system of embodiment 12, where the anodic electrochemical layer includes a an inorganic metal oxide electrochemically active material, such as WO₃, V₂O₅, MoO₃, Nb₂O₅, TiO₂, CuO, Ir₂O₃, Cr₂O₃, Co₂O₃, Mn₂O₃, Ta₂O₅, ZrO₂, HfO₂, Sb₂O₃, a lanthanide-based material with or without lithium, another lithium-based ceramic material, a nickel oxide (NiO, Ni₂O₃, or combination of the two), and Li, nitrogen, Na, H, or another ion, any halogen, or any combination thereof.

Embodiment 20. The method or system of embodiment 12, where the first transparent conductive layer includes indium oxide, indium tin oxide, doped indium oxide, tin oxide, doped tin oxide, zinc oxide, doped zinc oxide, ruthenium oxide, doped ruthenium oxide, silver, gold, copper, aluminum, and any combination thereof.

Note that not all of the activities described above in the general description or the examples are required, that a portion of a specific activity may not be required, and that one or more further activities may be performed in addition to those described. Still further, the order in which activities are listed is not necessarily the order in which they are performed.

Certain features that are, for clarity, described herein in the context of separate embodiments, may also be provided in combination in a single embodiment. Conversely, various features that are, for brevity, described in the context of a single embodiment, may also be provided separately or in any subcombination. Further, reference to values stated in ranges includes each and every value within that range.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

The specification and illustrations of the embodiments described herein are intended to provide a general understanding of the structure of the various embodiments. The specification and illustrations are not intended to serve as an exhaustive and comprehensive description of all of the elements and features of apparatus and systems that use the structures or methods described herein. Separate embodiments may also be provided in combination in a single embodiment, and conversely, various features that are, for brevity, described in the context of a single embodiment, may also be provided separately or in any subcombination. Further, reference to values stated in ranges includes each and every value within that range. Many other embodiments may be apparent to skilled artisans only after reading this specification. Other embodiments may be used and derived from the disclosure, such that a structural substitution, logical substitution, or another change may be made without departing from the scope of the disclosure. Accordingly, the disclosure is to be regarded as illustrative rather than restrictive.

What is claimed is:

1. A system, comprising:
one or more wearable devices configured to generate state information;
one or more non-light emitting, variable transmission devices;
a remote management system configured to prioritize the state information and send a prioritized state information; and
a control device configured to change a transmission state for the one or more non-light emitting, variable transmission devices in response to receiving the prioritized state information.

2. The system of claim 1, further comprising at least one non-light-emitting, variable transmission device, wherein:
the non-light-emitting, variable transmission device comprises a first electrochromic device having a first edge, a second electrochromic device having a second edge, and a third electrochromic device having a third edge and a fourth edge;
the first edge of the first electrochromic device is immediately adjacent to the third edge of the third electrochromic device, and the second edge of the second electrochromic device is immediately adjacent to the fourth edge of the third electrochromic device; and
for a first scene, where comparing transmission levels of the first, second, and third electrochromic devices, the first electrochromic device has a lowest transmission level, the second electrochromic device has a graded transmission level, and the third electrochromic device has a highest transmission level.

3. The system of claim 1, wherein changing the transmission state of the non-light-emitting, variable transmission device comprises changing from a first state to a second state, wherein the first state is full clear and the second state is a graded transmission level.

4. The system of claim 1, wherein changing the transmission state of the non-light-emitting, variable transmission device comprises changing from a first state to a second state, wherein the first state is full tint and the second state is a full clear transmission.

5. The system of claim 1, wherein changing the transmission state of the non-light-emitting, variable transmission device comprises changing from a first state to a second state, wherein the first state is a graded transmission level and the second state is a fully tinted transmission level.

6. The system of claim 1, wherein the prioritized state information comprises a blood pressure information, heartbeat information, perspiration information, respiration frequency, and body temperature.

7. A method of controlling a non-light emitting, variable transmission device, comprising:
receiving state information from at least one wearable device;
prioritizing the received state information; and
sending signals from a remote management system to a first controller in response to the received prioritized state information;
changing a first transmission state of a non-light-emitting, variable transmission device to a second transmission state for the non-light-emitting, variable transmission device in response to the signals received from the first controller.

8. The method of claim 7, wherein the remote management system is a wireless system.

9. The method of claim 7, further comprising sending a signal to the at least one wearable device for permission to access the state information stored on the at least one wearable device.

10. The method of claim 7, wherein the first transmission state of the non-light-emitting, variable transmission device is full tint and the second transmission state is a graded transmission level.

11. A system, comprising:
a first non-light-emitting, variable transmission device;
a first wearable device;
a first controller coupled and configured to select a first scene from a collection of scenes for a non-light emitting, variable transmission device; and
a management system that includes a logic element configured to:
receive state information;
prioritize the received state information; and
send signals to the first controller in response to input corresponding to prioritized state information.

12. The system of claim 11, wherein the non-light-emitting, variable transmission device comprises:
a first transparent conductive layer;
a second transparent conductive layer;
a cathodic electrochemical layer between the first transparent conductive layer and the second transparent conductive layer; and
an anodic electrochemical layer between the first transparent conductive layer and the second transparent conductive layer.

13. The method or system of claim 12, wherein the non-light-emitting, variable transmission device further comprises a substrate, wherein the first transparent conductive layer is on the substrate.

14. The method or system of claim 13, wherein the substrate comprises glass, sapphire, aluminum oxynitride, spinel, polyacrylic compound, polyalkene, polycarbonate, polyester, polyether, polyethylene, polyimide, polysulfone, polysulfide, polyurethane, polyvinylacetate, another suitable transparent polymer, co-polymer of the foregoing, float glass, borosilicate glass, or any combination thereof.

15. The method or system of claim 12, wherein the cathodic electrochemical layer comprises WO₃, V₂O₅, MoO₃, Nb₂O₅, TiO₂, CuO, Ni₂O₃, NiO, Ir₂O₃, Cr₂O₃, Co₃O₃, Mn₂O₃, mixed oxides (e.g., W—Mo oxide, W—V oxide), lithium, aluminum, zirconium, phosphorus, nitrogen, fluorine, chlorine, bromine, iodine, astatine, boron, a borate with or without lithium, a tantalum oxide with or without lithium, a lanthanide-based material with or without lithium, another lithium-based ceramic material, or any combination thereof.

16. The method or system of claim 12, further comprising an ion-conducting layer between the cathodic electrochemical layer and the anodic electrochemical layer.

17. The method or system of claim 16, wherein the ion-conducting layer comprises lithium, sodium, hydrogen, deuterium, potassium, calcium, barium, strontium, magnesium, oxidized lithium, Li₂WO₄, tungsten, nickel, lithium carbonate, lithium hydroxide, lithium peroxide, or an alkaline earth metal, transition metal, Zn, Ga, Ge, Al, Cd, In, Sn, Sb, Pb, Bi, B, Si, P, S, As, Se, Te, silicates, silicon oxides, tungsten oxides, tantalum oxides, niobium oxides, borates, aluminum oxides, lithium silicate, lithium aluminum silicate, lithium aluminum borate, lithium aluminum fluoride, lithium borate, lithium nitride, lithium zirconium silicate, lithium niobate, lithium borosilicate, lithium phosphosilicate, other lithium-based ceramic materials, lithium salts, and dopants including lithium, sodium, hydrogen, deuterium, potassium, calcium, barium, strontium, magnesium, or combinations thereof.

18. The method or system of claim 12, wherein the second transparent conductive layer comprises indium oxide, indium tin oxide, doped indium oxide, tin oxide, doped tin oxide, zinc oxide, doped zinc oxide, ruthenium oxide, doped ruthenium oxide and any combination thereof.

19. The method or system of claim 12, wherein the anodic electrochemical layer comprises a an inorganic metal oxide electrochemically active material, such as WO₃, V₂O₅, MoO₃, Nb₂O₅, TiO₂, CuO, Ir₂O₃, Cr₂O₃, Co₂O₃, Mn₂O₃, Ta₂O₅, ZrO₂, HfO₂, Sb₂O₃, a lanthanide-based material with or without lithium, another lithium-based ceramic material, a nickel oxide (NiO, Ni₂O₃, or combination of the two), and Li, nitrogen, Na, H, or another ion, any halogen, or any combination thereof.

20. The method or system of claim 12, wherein the first transparent conductive layer comprises indium oxide, indium tin oxide, doped indium oxide, tin oxide, doped tin oxide, zinc oxide, doped zinc oxide, ruthenium oxide, doped ruthenium oxide, silver, gold, copper, aluminum, and any combination thereof.
